(19) United States
(12) Patent Application Publication
(10) Pub. No.: US 2006/0045910 A1
(43) Pub. Date: Mar. 2, 2006

(54) Preserved fusogenic vesicles

(76) Inventor: William D. Ehringer, Charleston, IN (US)

Correspondence Address: EVAN LAW GROUP LLC, 566 WEST ADAMS, SUITE 350, CHICAGO, IL, 60661, US

(21) Appl. No.: 10/933,125

(22) Filed: Sep. 2, 2004

(51) Int. Cl. A61K 31/7012; A61K 9/127

(52) U.S. Cl. 424/450; 514/53

(57) ABSTRACT

Preserved fusogenic vesicles are disclosed that include a saccharide, a fusogen, and a first polar phospholipid that is a stable vesicle former. The preserved fusogenic vesicles have a fusion rate of at least 20 vesicle fusions per second when re-hydrated. Methods of preserving fusogenic vesicles also are disclosed. Unexpectedly, after re-hydration the preserved fusogenic vesicles may transfer substantially more ATP through a cell membrane than unpreserved fusogenic vesicles.

### BACKGROUND

Lipid vesicles have unilamellar or multilamellar exterior walls that enclose an internal space. The walls of the vesicles are formed by a bimolecular layer of one or more lipid components having polar heads and non-polar tails. In an aqueous (or polar) liquid, the polar heads of one layer orient outwardly to extend into the surrounding medium, and the non-polar tail portions of the lipids associate with each other, thus providing a polar surface and a non-polar core in the wall of the vesicle. Unilamellar vesicles have one such bimolecular layer, whereas multilamellar vesicles generally have multiple concentric, bimolecular layers. While the exterior wall of a vesicle shares some similarity to cell walls found in living organisms, vesicles are not natural living cells containing organelles, such as those from plants, animals, bacteria, and the like.

Previously, lipid vesicle research was directed to making vesicles as stable as possible. Stable vesicles resist fusion with themselves and with other entities, such as cell membranes. Because conventional vesicles were intended to function as stable carriers for pharmaceutical and diagnostic agents, stability was considered advantageous.

Work also has been directed to preserving stable vesicles for long term storage. Examples of this work may be found in U.S. Pat. No. 5,008,109 to Tin and U.S. Pat. No. 4,857,319 to Crowe et al. In Crowe, for example, stable vesicles having diameters from about 30 nm to less than about 200 nm were freeze-dried with a disaccharide preserving agent. Crowe was directed to preventing the fusion of stable vesicles during freeze-drying, stating that vesicles having a diameter between 100 and 200 nm loose stability and the internal contents during freeze-drying. Specifically, vesicles having diameters from 200 to 400 nm retained only about 40% of the internal contents after the disclosed freeze-drying and re-hydration process.

Unlike conventional stable vesicles, fusogenic vesicles, such as those described in U.S. 2003/0235611 A1, are designed to transport materials, such as adenosine triphosphate (ATP), directly through cell membranes. As shown in FIG. 1, fusogenic vesicles are unstable and undergo a six fold increase in radius within two hours of formation. As the vesicles increase in size by fusing with one another, their ability to transport material through cell membranes rapidly decreases. Such a short useful lifetime prevented the storage of fusogenic vesicles, and necessitated that they be formed immediately prior to use. The present invention provides preserved fusogenic vesicles and allows for the vesicles to be made and stored prior to use.

### SUMMARY

In one aspect, preserved fusogenic vesicles are disclosed that include a saccharide, a fusogen, and a first polar phospholipid that is a stable vesicle former. The stable vesicle former may form vesicles at least 50% of which persist for at least one hour, while the fusogen may include an unstable vesicle former. The preserved fusogenic vesicles have a fusion rate of at least 20 vesicle fusions per second when re-hydrated.

In another aspect, preserved fusogenic vesicles are disclosed that include a saccharide, a fusogen, and a first polar phospholipid that is a stable vesicle former. The preserved fusogenic vesicles have an average hydrodynamic diameter of at least 200 nm when re-hydrated.

In yet another aspect, a method for forming a mixture from which preserved fusogenic vesicles may be formed is disclosed. The method includes combining water, a saccharide, a fusogen, and a first polar phospholipid that is a stable vesicle former to form the mixture, where vesicles formed from the fusogen and the first polar phospholipid having an average hydrodynamic diameter from 250 nm to 350 nm have a fusion rate of at least 20 vesicle fusions/second.

In yet another aspect, a method for preserving fusogenic vesicles is disclosed that includes freeze-drying a composition that includes water, fusogenic vesicles, and a saccharide. The preserved fusogenic vesicles have a fusion rate of at least 20 vesicle fusions/second when re-hydrated.

The scope of the present invention is defined solely by the appended claims, and is not affected to any degree by the statements within this summary.

### BRIEF DESCRIPTION OF THE DRAWINGS

The invention can be better understood with reference to the following drawings and description. The components in the figures are not to scale, emphasis instead being placed upon illustrating the principles of the invention.

FIG. 1 is a graph showing the rapid rate at which fusogenic vesicles fuse with one another after formation.

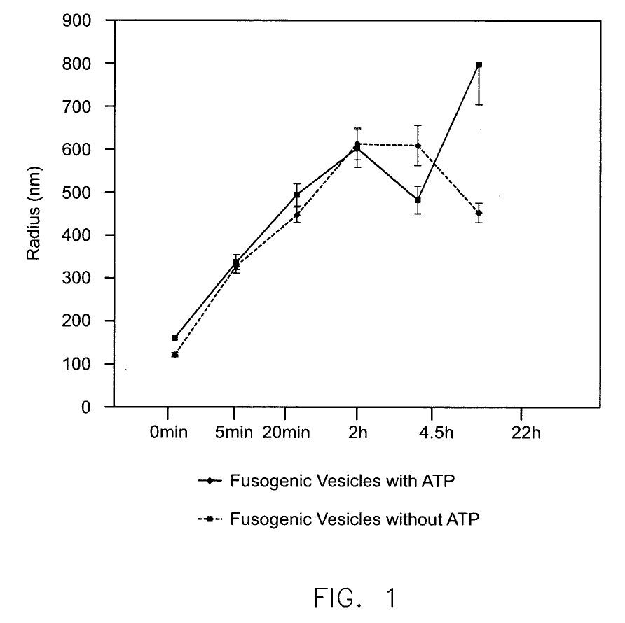

FIG. 2 is a graphical representation of a fusogenic vesicle. The figure is not intended to accurately represent an actual vesicle.

FIG. 3 provides a preferred method of preserving fusogenic vesicles.

FIG. 4 depicts the spread of membrane bilayer radii of preserved fusogenic vesicles obtained from a preferred preservation method.

FIG. 5 graphically depicts the superior uptake of ATP by rat cardiomyocyte cells from fusogenic vesicles preserved in accordance with the present invention.

FIG. 6 graphically depicts the average hydrodynamic radii of freshly prepared fusogenic vesicles and preserved and re-hydrated fusogenic vesicles taken from the same batch.

### DETAILED DESCRIPTION

The present invention makes use of the discovery that saccharides may be used to preserve fusogenic vesicles. Re-hydrated preserved fusogenic vesicles in accord with the present invention have fusion rates of at least 20 vesicle fusions per second. Unlike conventional stable vesicles, fusogenic vesicles have destabilized membrane bilayers. Thus, the very characteristic that allows fusogenic vesicles to pass ATP through cell membranes, their fusibility, limits their useful lifetime to less than about two hours without preservation.

The preserved fusogenic vesicles of the present invention have average hydrodynamic diameters that are significantly larger than those previously believed capable of preservation. For example, in Crowe stable vesicles having diameters of 200 nm and larger retained about 40% of the internal contents after preservation and re-hydration. Thus, approximately 60% of the 200 nm and larger vesicles preserved and re-hydrated by the method disclosed in Crowe lost internal contents or were rendered useless.

In contrast to Crowe, the preserved and re-hydrated fusogenic vesicles of the present invention retain at least 70%, preferably, at least 95% of their pre-preservation ATP transfer ability. Thus, the pre-preservation activity of the newly formed vesicles is substantially maintained or improved for the preserved and re-hydrated vesicles. This is especially surprising because the fusogenic vesicles preserved by the present invention are initially less stable than those described in Crowe.

In further contrast to Crowe, re-hydrated fusogenic vesicles preserved by the present invention may have nearly identical hydrodynamic diameters to freshly prepared fusogenic vesicles. The destruction of approximately 60% of the 200 nm and larger vesicles preserved and re-hydrated by Crowe would result in a substantial change in the average diameter of the re-hydrated vesicles in relation to their freshly prepared counterparts. Surprisingly, the preservation method of the present invention may provide re-hydrated vesicles having a nearly identical hydrodynamic diameter with only a slight distribution increase in relation to freshly prepared vesicles.

The following definitions are included to provide a clear and consistent understanding of the specification and claims.

“Alkyl” (or alkyl- or alk-) refers to a substituted or unsubstituted, straight, branched or cyclic hydrocarbon chain, preferably containing from 1 to 20 carbon atoms. Suitable examples of unsubstituted alkyl groups include methyl, ethyl, propyl, isopropyl, cyclopropyl, butyl, iso-butyl, tert-butyl, sec-butyl, cyclobutyl, pentyl, cyclopentyl, hexyl, cyclohexyl, and the like. “Alkylaryl” and “alkylheterocyclic” groups are alkyl groups covalently bonded to an aryl or heterocyclic group, respectively.

“Alkenyl” refers to a substituted or unsubstituted, straight, branched or cyclic, unsaturated hydrocarbon chain that contains at least one double bond, and from 2 to 20 carbon atoms. Exemplary unsubstituted alkenyl groups include ethenyl (or vinyl), 1-propenyl, 2-propenyl (or allyl) 1,3-butadienyl, hexenyl, pentenyl, 1,3,5-hexatrienyl, and the like. Preferred cycloalkenyl groups contain five to eight carbon atoms and at least one double bond. Examples of cycloalkenyl groups include cyclohexadienyl, cyclohexenyl, cyclopentenyl, cycloheptenyl, cyclooctenyl, cyclohexadienyl, cycloheptadienyl, cyclooctatrienyl and the like.

“Alkoxy” refers to an —OR group, where R is a substituted or unsubstituted alkyl group. Exemplary alkoxy groups include methoxy, ethoxy, n-propoxy, iso-propoxy, n-butoxy, t-butoxy, and the like.

“Aryl” refers to any monovalent aromatic carbocyclic or heteroaromatic group, preferably of 3 to 10 carbon atoms. The aryl group can be bicyclic (i.e., phenyl (or Ph)) or polycyclic (i.e., naphthyl) and can be unsubstituted or substituted. Preferred aryl groups include phenyl, naphthyl, furyl, thienyl, pyridyl, indolyl, quinolinyl or iso-quinolinyl.

“Amino” refers to an unsubstituted or substituted-NRR′ group, where R and R′ are independently selected from hydrogen and substituted or unsubstituted alkyl groups. The amine can be primary (—NH₂), secondary (—NHR), or tertiary (—NRR′), depending on the number of substituents (R or R′). Examples of substituted amino groups include methylamino, dimethylamino, ethylamino, diethylamino, 2-propylamino, 1-propylamino, di-(n-propyl) amino, di-(iso-propyl) amino, methyl-n-propylamino, t-butylamino, anilino, and the like.

“Substituted” means that the moiety contains at least one, preferably from 1 to 3 substituent(s). Suitable substituents include hydrogen (H) and hydroxyl (—OH), amino (—NH₂), oxy (—O—), carbonyl (—CO—), thiol, alkyl, alkenyl, alkynyl, alkoxy, halo, nitrile, nitro, aryl and heterocyclic groups. These substituents can optionally be further substituted with from 1 to 3 substituents. Examples of substituted substituents include carboxamide, alkylmercapto, alkylsulphonyl, alkylamino, dialkylamino, carboxylate, alkoxycarbonyl, alkylaryl, aralkyl, alkylheterocyclic, and the like.

A “mixture” is intended to include solutions, dispersions, suspensions, solid/liquid mixtures, and liquid/liquid mixtures. Solutions, unlike dispersions, suspensions, and mixtures, lack an identifiable interface between their solubilized molecules and the solvent. Hence, the term mixture may be used when a solid is in direct contact with a liquid (a solution) and when the solid is merely carried or suspended by the liquid. In either instance, the liquid may be referred to as a “solvent.”

The term “fusogenic” describes the ability of a vesicle to fuse with, thus becoming part of, a target cell membrane.

A “fusogen” is any substance that increases the ability of a lipid vesicle bilayer to fuse with, thus becoming part of, a target cell membrane. Upon fusing, the lipid vesicle may release the contents of the vesicle into the interior of the cell. Fusogens exclude stable vesicle formers and may destabilize the vesicle.

“Polar lipids” are organic molecules having a hydrophilic end (the “head”) joined by a backbone to a hydrophobic end (the “tail”). A “polar phospholipid” is a polar lipid having a phosphorous head group. In one aspect, polar phospholipids include at least six carbon atoms. Structure (I), shown below, depicts a preferred polar phospholipid where X is the head, L is the backbone, and Z is the tail. The two Z groups may be the same or different.
X-----L------Z₂ Structure (I)

The phosphorous containing head group X of the polar phospholipid is preferably represented by Structure (II), shown below, where B preferably is an alkyl group or a cation, such as Na⁺, K⁺, or CH₄N⁺. The dashed bond in each structure represents a bonding location, in this instance, the bond formed between phosphorous and the L group.

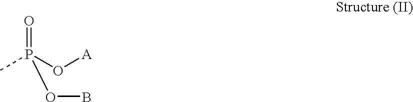

In one aspect, A is hydrogen or an alkyl group; preferably A is an alkyl group substituted with an amine. At present, A is more preferably a group having Structure (III), (IV), (V), (VI) or (VII), as shown below. Throughout this specification, the structures may show molecules in their protonated or deprotonated forms; however, the structures also are intended to include deprotonated and protonated forms, respectively. The form of the molecule present in the composition or the mixture at a specific time depends on the pH of the composition, the presence or absence of water, and/or the available counter ions.

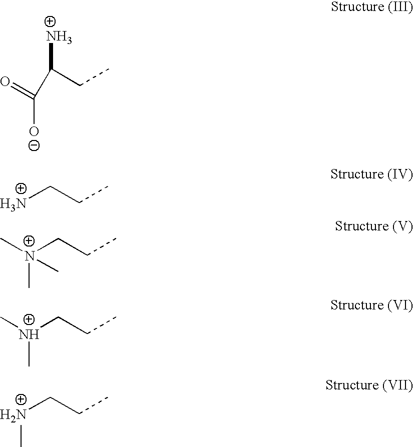

The backbone group L of the polar phospholipid represented by Structure (I) above may be any alkyl group having three or more substituents, with one of the substituents being an X group and the remaining two substituents being Z groups. In a preferred aspect, the alkyl group L is substituted with heteroatoms, such as with substituents having alkoxy or amino functionality that provide the connection to the X and two Z groups. In a preferred aspect, L is a group having Structure (VIII), (IX), or (X), as shown below.

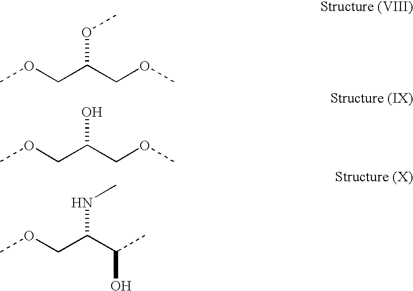

The tail groups Z may be the same or different and may be an alkyl or alkenyl group. The Z groups also may include a carbonyl group —(CO)— that links the L group to an alkyl or alkenyl group. In one aspect, the linked alkyl or alkenyl group is an unsubstituted straight chain having from 6 to 26 carbon atoms. In a preferred aspect, when the Z group includes a carbonyl group, the linked alky group is —C₁₅H₃₁ or —C₁₇H₃₅. In a preferred aspect, when the Z group includes a carbonyl group, the linked alkenyl is a group having Structure (XI), (XII), or (XIII), as shown below.

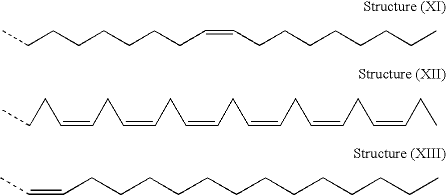

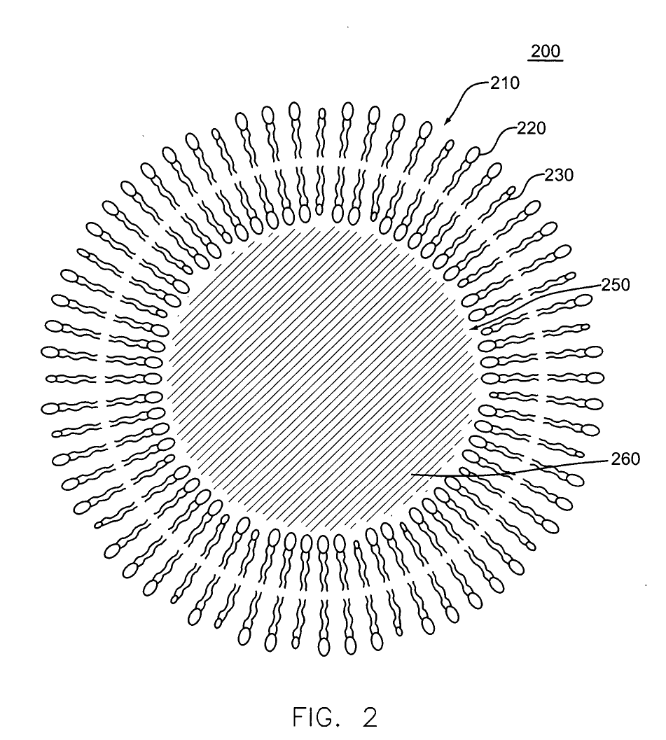

FIG. 2 is a graphical representation of a fusogenic vesicle 200. Fusogenic vesicles are vesicles that may rapidly fuse with themselves or preferably with cell membranes, such as the cell membrane of human umbilical vein endothelial cells (HUVECs). Vesicle fusion rate (fusogenicity) is a measure of the number of vesicles that fuse with the HUVEC cells in a well per second (about 10⁶ cells) and is determined by:
(1) loading prepared vesicles with a fluorescent probe, such as carboxyfluorescein;
(2) allowing the loaded vesicles to fuse with cultured HUVEC cells (American Type Culture Collection (ATCC); Manassus, Va. or BioWhittaker; Md.);
(3) removing any residual vesicles at a selected time; and
(4) determining the fluorescence of the HUVEC cells as a function of time.

The HUVEC cells are grown to confluence on 12-well culture dishes in endothelial cell growth medium and washed 3 times with a buffer, such as HBSS. The prepared vesicles are loaded with 1 mM carboxyfluorescein and incubated with the cells for 120 minutes at 37° C., 95% air/5% CO₂. The vesicles are then added to the HUVEC cells, thus initiating the fusion process. If negatively charged vesicles are used, calcium (final concentration 0.1-10 mM) is added at the fusion step.

At a selected time, the residual vesicles are removed from a well by washing the cells with buffer to quench the fusion reaction. The HUVEC cells then are removed from the well by treating with trypsin. The fluorescence of the collected cells may then be determined with a luminescence spectrophotometer or other suitable device (excitation at 495 nm and emission of 520 nm). By quenching the fusion reaction and determining the fluorescence of the HUVEC cells at selected time intervals, such as every 5 or 15 minutes, the rate at which the vesicles are delivering the carboxyfluorescein to the cells may be determined. Thus, the intensity of the fluorescent signal emitted by the HUVEC cells indicates the ability of the vesicles to fuse with the cell membranes and deliver their contents into the cells.

When determined as outlined above, the preferable fusion rate for preserved vesicles with HUVEC cells is at least 20 vesicle fusions per second when re-hydrated. More preferably, the fusion rate with HUVEC cells is at least 1×10⁵, at least 1×10¹⁰, or at least 1×10¹² fusions per second when re-hydrated. In another aspect, re-hydrated vesicles fuse at preferable rates from 20 to 8×10¹¹, from 7.5×10⁵ to 8×10⁸, from 1×10⁷ to 1×10⁸, or from 5×10⁶ to 1×10⁷ fusions per second. In an aspect especially preferred at present, the fusion rate is about 1×10¹⁴ fusions per second when re-hydrated. Unless stated otherwise, all vesicle fusion rates are presented in relation to HUVEC cells.

Re-hydration is performed by adding the preserved vesicles to the same amount of water as was removed during the prior freeze-drying process and gently mixing the resulting suspension, such as with a vortex mixer, for 10 minutes at 25° C. For example, if the original vesicle mixture included 25 mg of lipid material to 1 mL of water, then 25 mg of the preserved vesicles would be re-hydrated in 1 mL of water.

The fusogenic vesicle 200 includes a membrane bilayer 210 that encloses an internal space 250. The internal space 250 may contain an aqueous mixture or solution that includes one or a plurality of water soluble species, such as salts. At present, cationic salts, such as magnesium salts, of adenosine triphosphate (ATP) are preferably included in the aqueous solution. Molecules other than ATP may be delivered to cells using the fusogenic vesicle, such as organic and inorganic molecules, bioactive agents, pharmaceuticals, polypeptides, nucleic acids, and antibodies that interact with intracellular antigens.

The membrane bilayer 210 of the fusogenic vesicle 200 resembles a plasma membrane and may be tailored to fuse with a variety of cell membranes at different rates. The membrane bilayer 210 may have a tight radius of curvature, thus making the vesicle highly energetic. In one aspect, the average hydrodynamic diameter of the membrane bilayer 210 is from 20 to 450 nm, preferably from 150 to 400 nm, more preferably from 200 to 380 nm, and even more preferably from 250 to 350 nm. At present, the preferred average hydrodynamic diameter for the membrane bilayer 210 is about 300 nm.

These hydrodynamic diameters are believed to assist in allowing the membrane bilayer 210 to pass ATP through cell membranes and possibly through the gaps between endothelial cells. Useful vesicles may vary in average hydrodynamic diameter and may be selected according to a specific application. For example, if the rate at which a specific cell or tissue requires ATP is known, vesicle hydrodynamic diameter may be tailored to provide a vesicle fusion rate that delivers ATP at this approximate rate to the cell or tissue.

The average hydrodynamic diameter of the fusogenic vesicle 200 is defined as twice the average hydrodynamic radius of the membrane bilayer 210. In comparison to the diameter or average diameter of a vesicle, the average hydrodynamic diameter of the membrane bilayer 210 includes the water and ions associated with the outer surface of the bilayer 210. Thus, the hydrodynamic diameter of a specific vesicle is numerically larger than the diameter of that vesicle.

The average hydrodynamic diameter of a vesicle may be determined by Dynamic Light Scattering (DLS). DLS may be performed by directing a laser at an aqueous sample that includes the vesicles, while measuring the light scattered by the vesicles. The intensity of the light scattered by the vesicles may be measured with a photometer oriented 90° relative to the light source. As the vesicles move in the aqueous sample, the intensity of the light scattered by the vesicles changes over a given time period. From the light intensity data gathered as a function of time from the photometer, the hydrodynamic radius and/or diameter of the membrane bilayer 210, including any associated water and ions that solvate the membrane, may be determined. DLS measurements may be obtained using a Proterion DynaPro Dynamic Light Scattering Instrument, available from Proterion Co., Piscataway, N.J.

The membrane bilayer 210 may include a first polar phospholipid 220 that is a “stable vesicle former.” Stable vesicle formers are polar phospholipids that will form vesicles at least 50% of which will persist for at least one hour, when prepared as follows: first, the phospholipid is dissolved in chloroform and placed in a glass test tube. The chloroform is then removed by evaporation under a steady stream of nitrogen, followed by vacuum for twelve hours. The dried lipid material is then re-hydrated in 10 mM Na₂HPO₄ to give a 25 mg/mL concentration. The resultant aqueous mixture is maintained for 60 minutes at a temperature above the phase transition temperature of the lipid. The lipid vesicles are then reduced in size by any convenient means, such as by high pressure homogenization or by sonication with a micro-tip 450 watt sonicator used at a 40% duty cycle.

Lipids that may be used as the first polar phospholipid 220 include Soy Phosphatidylcholine (SOYPC) (Structure (XIV), dioleoylphosphatidylcholine (DOPC) (Structure (XV)), 1-palmitoyl-2-docosahexaenoyl-sn-glycero-3-phosphocholine (16:0,22:6 PC) (Structure (XVI)), 1-palmitoyl-2-oleoyl-phosphocholine (16:0,18:1 PC), 1-palmitoyl-2-linolinoyl-3-phosphocholine (16:0,18:3 PC), 1-palmitoyl-2-arachidonoyl-3-phosphocholine (16:0, 20:4, PC), or combinations thereof. Presently preferred lipids for use as the first polar phospholipid 220 include SOYPC and DOPC, with SOYPC being more preferred.

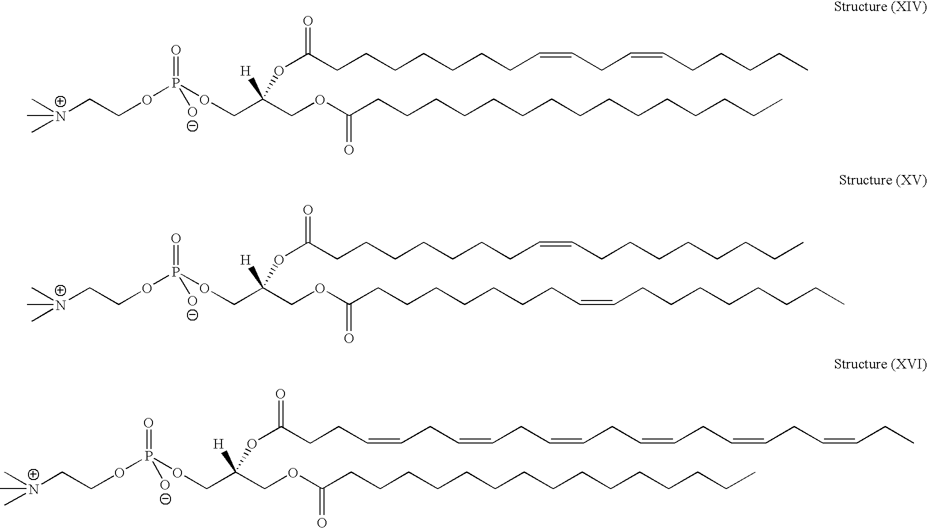

The membrane bilayer 210 includes a fusogen. The fusogen may not be phosphatidyl serine. Suitable fusogens include free fatty acids, aggregating agents, and “unstable vesicle formers.” Unstable vesicle formers, such as second polar lipid 230, are polar lipids that will not form vesicles at least 50% of which persist for at least one hour, when prepared as described for stable vesicle formers.

The fusogen may increase the rate of vesicle fusion by any pathway, including destabilizing and/or altering the surface charge of the membrane bilayer 210. In one aspect, and as represented in FIG. 2, the polar head group of the second polar lipid 230 is selected to be a “poor fit” with the polar head group of the first polar phospholipid 220, thus creating packing inefficiency between the polar phospholipids and destabilizing the membrane bilayer 210. Similarly, poorly fitting tails can also destabilize the membrane bilayer 210. In another aspect, the second polar lipid 230 may be selected to create a surface charge of opposite polarity to the charge of the cell membrane on the membrane bilayer 210 of the fusogenic vesicle 200.

Free fatty acids may be utilized as fusogens. In one aspect, free fatty acids such as oleic, stearic, palmitic, linoleic, linolenic, arachidonic, eicosopentaenoic, docosahexaenoic, or combinations thereof are preferred. At present, oleic acid (OA) is a preferred free fatty acid fusogen.

Aggregating agents also may be utilized as fusogens. Useful aggregating agents may include water absorbing materials that include polyethylene glycol (PEG); salts of divalent metals, such a Ca²⁺ and/or Mg²⁺; polymers, such as hydroxyethylstarch; and mixtures thereof. In one aspect, PEG having a weight average molecular weight from 1,500 to 12,000 is preferred. At present, PEG having a weight average molecular weight of about 3,350 is preferred.

Polar lipids that may be used as the unstable vesicle former include Lyso-Phosphatidylcholine (Lyso-PC), 1,2-dioleoyl-sn-glycero-3-ethylphosphocholine (DOPC-e), 1-palmitoyl-2-oleyl-3-glycerophosphorcholine (POPA), 1,2-dioleoyl-3-trimethylammoniumpropane (DOTAP), 1-steroyl-2-docosaheaxenoyl-3-phosphocholine (18:0, 22:6, PC), mixed chain phosphatidyl choline (MPC), phosphatidyl ethanol (PE), 1,2-dioleoyl-sn-glycero-3-phosphoethanolamine (DOPE), 1-palmitoyl-2-hydroxy-sn-glycero-3-phosphocholine (16:0-Lyso PC), or combinations thereof. Presently, preferred polar lipids for use as the unstable vesicle former include Lyso-PC, DOPC-e, POPA, or DOTAP. In one aspect, a combination of Lyso-PC and free fatty acids is a preferred fusogen.

In order to tune the fusogenicity of the fusogenic vesicle 200, the ratio of the first polar phospholipid 220 to the fusogen may be altered. In one aspect, about 5% of a fusogen including oleic acid and Lyso-PC may be combined with 95% of the first polar phospholipid 220 on a weight/weight basis (w/w). In another aspect, the first polar phospholipid 220 may be combined with the second polar lipid 230 in a molar ratio (m/m) from 500:1 to 1:1 or from 100:1 to 10:1. At present, a molar ratio from 60:1 to 15:1 or about 50:1 (m/m) is preferred.

The membrane bilayer 210, and other portions of the fusogenic vesicle 200, such as the internal space 250, may include a saccharide. Thus, the internal and external surfaces of the membrane bilayer 210 may be coated by the saccharide, while the internal space 250 may contain the saccharide. The saccharide may be incorporated into the fusogenic vesicle 200 at a ratio (m/m) with the first polar lipid 220 from 5:1 to 1:5. At present, a ratio of about 1:1 is preferred.

Preferably, the saccharide is any water-soluble saccharide, including monosaccharide, disaccharide, and polysaccharide. The saccharide also may include enantiomers, diastereomers, derivatives, and racemic mixtures of one or more saccharides, which are capable of preserving the fusogenic vesicle, while maintaining the desired fusogenicity. While not wishing to be bound by any particular theory, it is believed that the saccharide prevents vesicle fusion during the dehydration process by binding to the polar head groups of the lipids, displacing water, and creating a glass that surrounds and protects the bilayer membrane from auto-fusion. Upon re-hydration, the saccharide is likely released, thus allowing water stabilization of the bilayer.

Preferable monosaccharides may include mannose, fructose, or ribose, but preferably not glucose. Preferable disaccharides may include trehalose, lactose, maltose, sucrose, or turanose. Preferable polysaccharides may include hydroxyethylstarch, inul in, or dextran. At present, a preferred saccharide is the disaccharide D-trehalose.

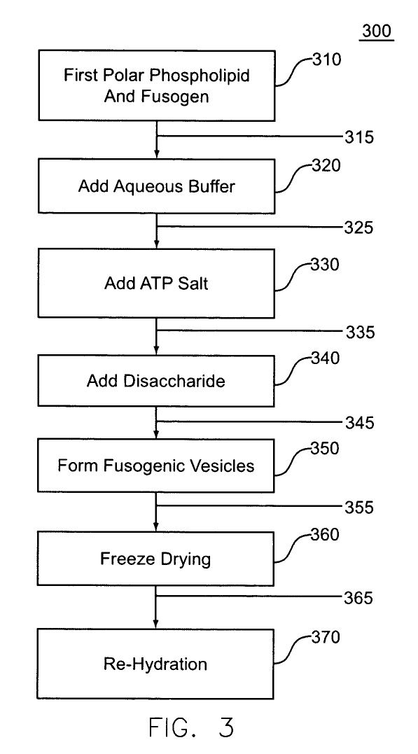

FIG. 3 depicts a method 300 of preserving fusogenic vesicles. In 310, an organic solvent, such as chloroform, is removed from a first polar phospholipid. The first polar phospholipid may be combined with a fusogen, such as a second polar lipid. In 320, an aqueous buffer is added to the dried first polar phospholipid and fusogen to form a first aqueous mixture 325. In 330, a salt of adenosine triphosphate is added to the first aqueous mixture 325 to form a second aqueous mixture 335. In 340, a saccharide is added to the second aqueous mixture 335 to form a third aqueous mixture 345. At least a portion of any non-hydrated lipids optionally may be removed from the third aqueous mixture 345.

In 350, the third aqueous mixture 345 may be mixed by any technique that results in fusogenic vesicles having the desired fusion rate. Suitable mixing techniques may include sonication, homogenization, static mixing, extrusion, such as through a microporous membrane, or combinations thereof. The resulting fourth aqueous mixture 355 optionally may be “snap-frozen,” such as in liquid nitrogen, prior to freeze-drying.

In 360, the fourth aqueous mixture 355 is freeze-dried (lyophilized) to form preserved fusogenic vesicles having a fusion rate of at least 20 vesicle fusions per second after re-hydration 370. The temperature at which the freeze drying 360 is performed is preferably below the freezing point of the fourth aqueous mixture 355. For example, when trehalose is the saccharide, a freeze drying temperature of −40° C. and below, more preferably −42° C. and below, may be used.

Prior to the re-hydration 370, a storage period 365 may be from 10 minutes to 5 years, from 1 day to 2 years, or about 1 year. In one aspect, the vesicles re-hydrated in 370 retain at least 95% of the ability of newly formed fusogenic vesicles to pass ATP through cell membranes.

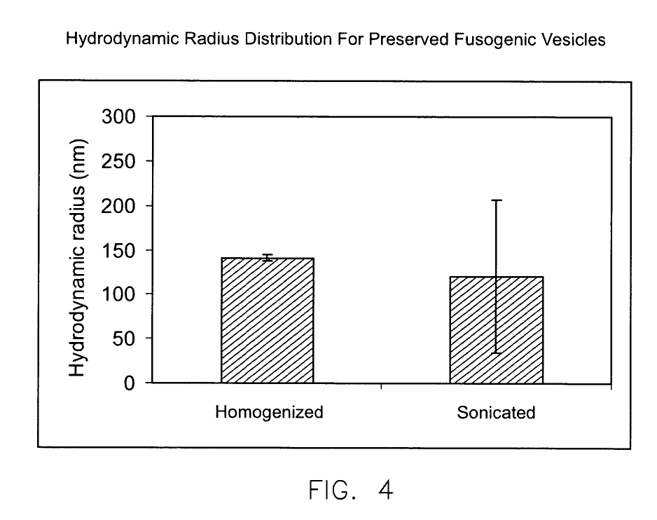

FIG. 4 depicts the average hydrodynamic radius of preserved fusogenic vesicles obtained from one embodiment of the preservation method 300. When the aqueous mixture 345 was mixed in 350 (FIG. 3) by homogenization, as opposed to sonication, less variation in the average hydrodynamic diameter of the resulting vesicles was observed. In the case of homogenization, preserved fusogenic vesicles having an average hydrodynamic diameter of 280±7.2 nm were obtained. Preserved fusogenic vesicles having other hydrodynamic diameters may be produced by altering the homogenization process.

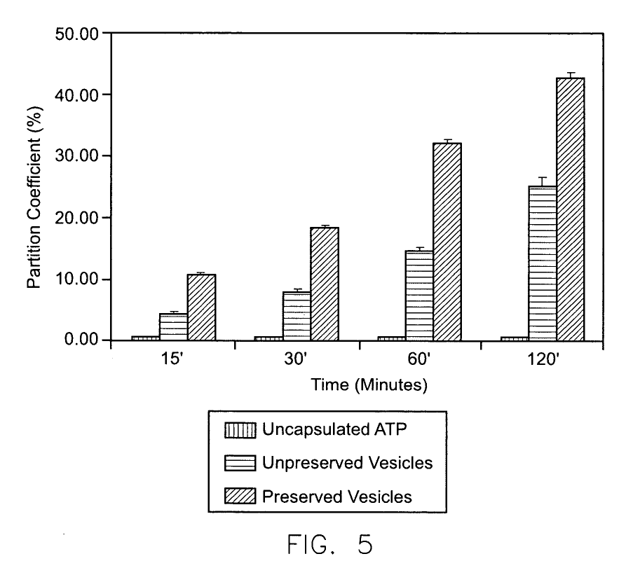

FIG. 5 graphically depicts the superior uptake of ATP by rat cardiomyocyte cells from fusogenic vesicles preserved in accordance with the present invention. From the graph it is clear that unencapsulated ATP is barely absorbed by the cells while the cells readily absorb ATP encapsulated in fusogenic vesicles. The preserved and re-hydrated fusogenic vesicles demonstrated an unexpectedly superior ability to transfer ATP through the cell membrane. In multiple instances, more than twice as much ATP was absorbed by the cells from the preserved vesicles than from the freshly formed vesicles. While not wishing to be bound by any specific theory, it is currently believed that the dehydration of the vesicles that occurs during freeze-drying causes more ATP to enter the fusogenic vesicles. In this manner, the preserved and re-hydrated fusogenic vesicles may contain a higher concentration of ATP than their unpreserved counterparts.

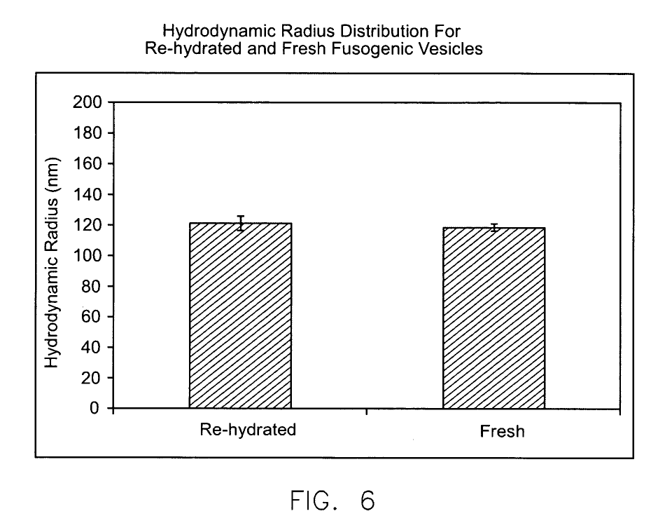

FIG. 6 graphically depicts that the average hydrodynamic radius of freshly prepared fusogenic vesicles and preserved and re-hydrated fusogenic vesicles taken from the same batch retain nearly identical average hydrodynamic radii. In one aspect, the freshly prepared vesicles had an average hydrodynamic diameter of 236.8±4.6 nm while their preserved and re-hydrated counterparts had an average hydrodynamic diameter of 242.2±9.4 nm. Thus, the preservation and re-hydration processes of the present invention resulted in only a slight increase in the distribution of vesicle diameter.

This result suggests that the preservation method of the present invention does not markedly alter the physical structure of the freshly prepared vesicles when the preserved vesicles are re-hydrated.

### EXAMPLES

### Example 1

### Formation of Preserved Fusogenic Vesicles

A mixture containing approximately 95 weight percent Soy Phosphatidylcholine (SOYPC) and approximately 5 weight percent (w/w) of a 1:1 mixture of lysophosphatidylcholine (Lyso-PC) and free fatty acids, including oleic acid, and combined with 1,2-dioleoly-sn-glycero-3-ethylphosphocholine (DOPC-e) in a 1:50 m/m ratio of DOPC-e to SOYPC in chloroform (˜20 mg lipids to 1 mL chloroform). The lipids were obtained from Avanti Polar Lipids (Alabaster, Ala.) and were combined without further purification. After dissolving the lipids in chloroform, the chloroform was removed by evaporation under a steady stream of nitrogen gas, followed by overnight vacuum pumping.

The dried lipid material was re-hydrated in HBSS aqueous experimental buffer (Sigma; St. Louis, Mo.) at about 25° C. for 30 minutes. Mg-ATP was added to the aqueous mixture until a 5 mM solution concentration was reached. D-(+)-trehalose (Ferro-Pfanstiehl; Cleveland, Ohio) was added on a 1:1 molar basis with the SOYPC. Two glass beads were added to the buffer/ATP/lipid mixture, and the mixture was vortexed for five minutes to create multilamellar vesicles. The resulting mixture was then sonicated using the micro-tip of a Branson Sonifier 450 (Branson Sonifiers; UK). The vesicles were then sonicated for five minutes at level 5 with a 40% duty cycle to create small unilamellar vesicles (SUVs). If necessary, the pH of the solution is adjusted to between 7.3 and 7.4.

The test tubes containing the vesicles were then transferred to a swinging bucket centrifuge and the tubes were centrifuged on high for 5-8 minutes to remove titanium particles and any non-hydrated lipids. The supernatant was carefully removed from the tubes without disturbing the titanium or particle bed (either by leaving approx. 1 mL of lipid in the tube or by filtering through a 0.2 μm syringe filter). The supernatant containing the vesicles was then snap-frozen in liquid nitrogen. The frozen vesicles were then freeze-dried on a Labconco lyophilizer overnight or longer at a vacuum of 130 mBar or below.

### Example 2

### Additional Lipids from Which Preserved Fusogenic Vesicles were Formed

The general method of Example 1 was used to form preserved fusogenic vesicles from a lipid system that included 1,2-dioleoyl-sn-glycero-3-phosphocholine (DOPC) and 1-palmitoyl-2-oleoyl-sn-glycero-3-phosphate (POPA) lipids in a 50:1 molar ratio.

The general method of Example 1 was modified so the SOYPC/OA/Lyso-PC lipid combination (95% Soy phosphatidylcholine with 5% lysophosphatidylcholine/oleic acid) was initially combined with 20 mole % polyethylene glycol (PEG-3350) in chloroform.

As any person of ordinary skill in the art of vesicle formation will recognize from the provided description, figures, and examples, modifications and changes can be made to the preferred embodiments of the invention without departing from the scope of the invention defined by the following claims and their equivalents.

What is claimed is:

1. Preserved vesicles, comprising:
a saccharide,
a fusogen, and
a first polar phospholipid that is a stable vesicle former,
where the preserved vesicles have a fusion rate of at least 20 vesicle fusions/second when re-hydrated.

2. The vesicles of claim 1, where the vesicle further comprises adenosine triphosphate.

3. The vesicles of claim 1, where the saccharide is selected from the group consisting of glucose, mannose, fructose, ribose, and combinations thereof.

4. The vesicles of claim 1, where the saccharide comprises a disaccharide.

5. The vesicles of claim 4, where the saccharide is selected from the group consisting of trehalose, lactose, maltose, sucrose, turanose, and combinations thereof.

6. The vesicles of claim 1, where the saccharide is selected from the group consisting of hydroxyethylstarch, inulin, dextran, and combinations thereof.

7. The vesicles of claim 1, where the saccharide is selected from the group consisting of trehalose, lactose, maltose, sucrose, mannose, turanose, and combinations thereof.

8. The vesicles of claim 1, where the saccharide comprises D-trehalose.

9. The vesicles of claim 1, where the ratio of the first polar phospholipid to the saccharide is from 5:1 to 1:5 (m/m).

10. The vesicles of claim 1, where the ratio of the first polar phospholipid to the saccharide is about 1:1 (m/m).

11. The vesicles of claim 1, where the first polar phospholipid is selected from the group consisting of soy phosphatidylcholine (SOYPC), dioleoylphosphatidylcholine (DOPC), 1-pal mitoyl-2-docosahexaenoyl-sn-glycero-3-phosphocholine (16:0,22:6 PC), 1-palmitoyl-2-oleoyl-phosphocholine (16:0,18:1 PC), 1-palmitoyl-2-linolinoyl-3-phosphocholine (16:0, 18:3 PC), 1-palmitoyl-2-arachidonoyl-3-phosphocholine (16:0, 20:4, PC), and mixtures thereof.

12. The vesicles of claim 1, where the first polar phospholipid includes soy phosphatidylcholine (SOYPC).

13. The vesicles of claim 1, where the first polar phospholipid includes dioleoylphosphatidylcholine (DOPC).

14. The vesicles of claim 1, where the fusogen is selected from the group consisting of a free fatty acid, an aggregating agent, an unstable vesicle former, and combinations thereof.

15. The vesicles of claim 1, where the fusogen comprises oleic acid.

16. The vesicles of claim 1, where the fusogen is selected from the group consisting of polyethylene glycol (PEG), hydroxyethylstarch, and combinations thereof.

17. The vesicles of claim 1, where the fusogen comprises polyethylene glycol having a weight average molecular weight from 1,500 to 12,000.

18. The vesicles of claim 1, where the fusogen comprises polyethylene glycol having a weight average molecular weight of about 3,350.

19. The vesicles of claim 1, where the fusogen comprises a second polar lipid.

20. The vesicles of claim 19, where the second polar lipid is selected from the group consisting of Lyso-Phosphatidylcholine (Lyso-PC), 1,2-dioleoyl-sn-glycero-3-ethylphosphocholine (DOPC-e), 1-palmitoyl-2-oleyl-3-glycerophosphorcholine (POPA), 1,2-dioleoyl-3-trimethylammoniumpropane (DOTAP), 1-steroyl-2-docosaheaxenoyl-3-phosphocholine (18:0, 22:6, PC), mixed chain phosphatidyl choline (MPC), phosphatidyl ethanol (PE), 1,2-dioleoyl-sn-glycero-3-phosphoethanolamine (DOPE), 1-palmitoyl-2-hydroxy-sn-glycero-3-phosphocholine (16:0-Lyso PC), and combinations thereof.

21. The vesicles of claim 19, where the second polar lipid is selected from the group consisting of Lyso-Phosphatidylcholine (Lyso-PC), 1,2-dioleoyl-sn-glycero-3-ethylphosphocholine (DOPC-e), 1-palmitoyl-2-oleyl-3-glycerophosphorcholine (POPA), 1,2-dioleoyl-3-trimethylammoniumpropane (DOTAP), and combinations thereof.

22. The vesicles of claim 19, where the second polar lipid comprises 1,2-dioleoyl-sn-glycero-3-ethylphosphocholine (DOPC-e).

23. The vesicles of claim 19, where the second polar lipid comprises 1-palmitoyl-2-oleyl-3-glycerophosphorcholine (POPA).

24. The vesicles of claim 19, where the second polar lipid comprises 1,2-dioleoyl-3-trimethylammoniumpropane (DOTAP).

25. The vesicles of claim 19, where the ratio of the first polar phospholipid to the second polar lipid is from 500:1 to 1:1 (m/m).

26. The vesicles of claim 19, where the ratio of the first polar phospholipid to the second polar lipid is from 100:1 to 10:1 (m/m).

27. The vesicles of claim 19, where the ratio of the first polar phospholipid to the second polar lipid is from 60:1 to 15:1 (m/m).

28. The vesicles of claim 1, where the first polar phospholipid comprises soy phosphatidylcholine (SOYPC) and the fusogen comprises Lyso-Phosphatidylcholine (Lyso-PC) and oleic acid.

29. The vesicles of claim 1 having an average hydrodynamic diameter from 20 nm to 450 nm.

30. The vesicles of claim 1 having an average hydrodynamic diameter from 250 nm to 350 nm.

31. The vesicles of claim 1 having an average hydrodynamic diameter of about 300 nm.

32. The vesicles of claim 1 having a fusion rate of at least 1×10¹⁰ vesicle fusions/second when re-hydrated.

33. Preserved vesicles, comprising
a saccharide,
a fusogen, and
a first polar phospholipid that is a stable vesicle former,
where the preserved vesicles have an average hydrodynamic diameter of at least 200 nm when re-hydrated.

34. The vesicles of claim 33, where the saccharide is selected from the group consisting of glucose, mannose, fructose, ribose, and combinations thereof.

35. The vesicles of claim 33, where the saccharide comprises a disaccharide.

36. The vesicles of claim 35, where the saccharide is selected from the group consisting of trehalose, lactose, maltose, sucrose, turanose, and combinations thereof.

37. The vesicles of claim 33, where the saccharide is selected from the group consisting of hydroxyethylstarch, inulin, dextran, and combinations thereof.

38. The vesicles of claim 33, where the saccharide is selected from the group consisting of trehalose, lactose, maltose, sucrose, mannose, turanose, and combinations thereof.

39. The vesicles of claim 33, where the saccharide comprises D-trehalose.

40. The vesicles of claim 33, where the ratio of the first polar phospholipid to the saccharide is from 5:1 to 1:5 (m/m).

41. The vesicles of claim 33, where the ratio of the first polar phospholipid to the saccharide is about 1:1 (m/m).

42. The vesicles of claim 33, where the first polar phospholipid is selected from the group consisting of soy phosphatidylcholine (SOYPC), dioleoylphosphatidylcholine (DO PC), 1-palmitoyl-2-docosahexaenoyl-sn-glycero-3-phosphocholine (16:0,22:6 PC), 1-palmitoyl-2-oleoyl-phosphocholine (16:0,18:1 PC), 1-palmitoyl-2-linolinoyl-3-phosphocholine (16:0, 18:3 PC), 1-palmitoyl-2-arachidonoyl-3-phosphocholine (16:0, 20:4, PC), and mixtures thereof.

43. The vesicles of claim 33, where the first polar phospholipid includes soy phosphatidylcholine (SOYPC).

44. The vesicles of claim 33, where the first polar phospholipid includes dioleoylphosphatidylcholine (DOPC).

45. The vesicles of claim 33, where the fusogen is selected from the group consisting of a free fatty acid, an aggregating agent, an unstable vesicle former, and combinations thereof.

46. The vesicles of claim 33, where the fusogen comprises oleic acid.

47. The vesicles of claim 33, where the fusogen is selected from the group consisting of polyethylene glycol (PEG), hydroxyethylstarch, and combinations thereof.

48. The vesicles of claim 33, where the fusogen comprises polyethylene glycol having a weight average molecular weight from 1,500 to 12,000.

49. The vesicles of claim 33, where the fusogen comprises polyethylene glycol having a weight average molecular weight of about 3,350.

50. The vesicles of claim 33, where the fusogen comprises a second polar lipid.

51. The vesicles of claim 50, where the second polar lipid is selected from the group consisting of Lyso-Phosphatidylcholine (Lyso-PC), 1,2-dioleoyl-sn-glycero-3-ethylphosphocholine (DOPC-e), 1-palmitoyl-2-oleyl-3-glycerophosphorcholine (POPA), 1,2-dioleoyl-3-trimethylammoniumpropane (DOTAP), 1-steroyl-2-docosaheaxenoyl-3-phosphocholine (18:0, 22:6, PC), mixed chain phosphatidyl choline (MPC), phosphatidyl ethanol (PE), 1,2-dioleoyl-sn-glycero-3-phosphoethanolamine (DOPE), 1-palmitoyl-2-hydroxy-sn-glycero-3-phosphocholine (16:0-Lyso PC), and combinations thereof.

52. The vesicles of claim 50, where the second polar lipid is selected from the group consisting of Lyso-Phosphatidylcholine (Lyso-PC), 1,2-dioleoyl-sn-glycero-3-ethylphosphocholine (DOPC-e), 1-palmitoyl-2-oleyl-3-glycerophosphorcholine (POPA), 1,2-dioleoyl-3-trimethylammoniumpropane (DOTAP), and combinations thereof.

53. The vesicles of claim 50, where the second polar lipid comprises 1,2-dioleoyl-sn-glycero-3-ethylphosphocholine (DOPC-e).

54. The vesicles of claim 50, where the second polar lipid comprises 1-palmitoyl-2-oleyl-3-glycerophosphorcholine (POPA).

55. The vesicles of claim 50, where the second polar lipid comprises 1,2-dioleoyl-3-trimethylammoniumpropane (DOTAP).

56. The vesicles of claim 50, where the ratio of the first polar phospholipid to the second polar lipid is from 500:1 to 1:1 (m/m).

57. The vesicles of claim 50, where the ratio of the first polar phospholipid to the second polar lipid is from 100:1 to 10:1 (m/m).

58. The vesicles of claim 50, where the ratio of the first polar phospholipid to the second polar lipid is from 60:1 to 15:1 (m/m).

59. The vesicles of claim 50, where the first polar phospholipid comprises soy phosphatidylcholine (SOYPC) and the fusogen comprises Lyso-Phosphatidylcholine (Lyso-PC) and oleic acid.

60. The vesicles of claim 50, where the preserved vesicles have an average hydrodynamic diameter of at least 250 nm.

61. The vesicles of claim 50, where the preserved vesicles have an average hydrodynamic diameter from 250 nm to 350 nm.

62. The vesicles of claim 50 having a fusion rate of at least 20 vesicle fusions/second when re-hydrated.

63. The vesicles of claim 50 having a fusion rate of at least 1×10¹⁰ vesicle fusions/second when re-hydrated.

64. The vesicles of claim 50 having a fusion rate of at least 1×10¹² vesicle fusions/second when re-hydrated.

65. A method for forming a mixture to provide preserved fusogenic vesicles, comprising:
combining water, a saccharide, a fusogen, and a first polar phospholipid that is a stable vesicle former to form the mixture,
where vesicles formed from the fusogen and the first polar phospholipid having an average hydrodynamic diameter from 250 nm to 350 nm have a fusion rate of at least 20 vesicle fusions/second.

66. The method of claim 65, where the preserved fusogenic vesicles formed from the mixture have an average hydrodynamic diameter from 20 nm to 450 nm.

67. The method of claim 65, where the vesicles formed from the fusogen and the first polar phospholipid have a fusion rate of at least 1×10¹⁰ vesicle fusions/second.

68. The method of claim 65, further comprising combining ATP into the mixture.

69. The method of claim 65, where the saccharide comprises a disaccharide

70. The method of claim 65, where the saccharide comprises D-trehalose.

71. The method of claim 65, where the ratio of the first polar phospholipid to the saccharide is from 5:1 to 1:5 (m/m) in the mixture.

72. The method of claim 65, where the first polar phospholipid is selected from the group consisting of soy phosphatidylcholine (SOYPC), dioleoylphosphatidylcholine (DOPC), 1-palmitoyl-2-docosahexaenoyl-sn-glycero-3-phosphocholine (16:0,22:6 PC), 1-palmitoyl-2-oleoyl-phosphocholine (16:0,18:1 PC), 1-palmitoyl-2-linolinoyl-3-phosphocholine (16:0, 18:3 PC), 1-palmitoyl-2-arachidonoyl-3-phosphocholine (16:0, 20:4, PC), and mixtures thereof.

73. The method of claim 65, where the fusogen is selected from the group consisting of a free fatty acid, an aggregating agent, an unstable vesicle former, and combinations thereof.

74. The method of claim 65, where the ratio of the first polar phospholipid to the second polar lipid is from 500:1 to 1:1 (m/m) in the mixture.

75. A method for preserving fusogenic vesicles, comprising:
freeze-drying a composition comprising water, vesicles, and a saccharide to give preserved fusogenic vesicles, where
the preserved fusogenic vesicles have a fusion rate of at least 20 vesicle fusions/second when re-hydrated.

76. The method of claim 75, where the fusogenic vesicles comprise a fusogen selected from the group consisting of a free fatty acid, an aggregating agent, an unstable vesicle former, and combinations thereof.

77. The method of claim 75, where the fusogenic vesicles comprise a first polar phospholipid selected from the group consisting of soy phosphatidylcholine (SOYPC), dioleoylphosphatidylcholine (DOPC), 1-palmitoyl-2-docosahexaenoyl-sn-glycero-3-phosphocholine (16:0,22:6 PC), 1-palmitoyl-2-oleoyl-phosphocholine (16:0,18:1 PC), 1-palmitoyl-2-linolinoyl-3-phosphocholine (16:0, 18:3 PC), 1-palmitoyl-2-arachidonoyl-3-phosphocholine (16:0, 20:4, PC), and mixtures thereof.

78. The method of claim 75, where the fusogenic vesicles comprise adenosine triphosphate.

79. The method of claim 75, further comprising forming the fusogenic vesicles by a technique selected from the group consisting of sonication, homogenization, static mixing, extrusion, and combinations thereof.

80. The method of claim 75, further comprising snap-freezing the composition before the freeze-drying.

81. The method of claim 75, where the freeze-drying is performed below the freezing point of the composition.
